(19) United States
(12) Patent Application Publication
(10) Pub. No.: US 2016/0279058 A1
(43) Pub. Date: Sep. 29, 2016

(54) PROTEIN-BASED GEL DELIVERY SYSTEM

(71) Applicants: Cesar Rodriguez, Fort Worth, TX (US); David Henzler, Fort Worth, TX (US); Megha Sharma, Fort Worth, TX (US); Alan Jones, Fort Worth, TX (US)

(72) Inventors: Cesar Rodriguez, Fort Worth, TX (US); David Henzler, Fort Worth, TX (US); Megha Sharma, Fort Worth, TX (US); Alan Jones, Fort Worth, TX (US)

(21) Appl. No.: 15/079,852

(22) Filed: Mar. 24, 2016

Related U.S. Application Data
(60) Provisional application No. 62/137,289, filed on Mar. 24, 2015.

(51) Int. Cl. A61K 9/06; A61K 47/46; A61K 47/42; A61K 45/06; A61K 31/355; A61K 31/4188; A61K 31/593; A61K 31/07; A61K 31/122; A61K 31/519; A61K 31/197; A61K 31/714; A61K 31/51; A61K 31/525; A61K 31/4415; A61K 31/455; A61K 9/00

(52) U.S. Cl. CPC A61K 9/06; A61K 9/0053; A61K 47/46; A61K 47/42; A61K 45/06; A61K 31/355; A61K 31/4188; A61K 31/593; A61K 31/07; A61K 31/122; A61K 31/519; A61K 31/197; A61K 31/714; A61K 31/51; A61K 31/525; A61K 31/4415; A61K 31/455; A23L 1/0562; A23L 1/296; A23L 1/1643; A23L 1/302; A23L 1/303; A23L 1/05625; A23L 1/304; A23V 2002/00

(57) ABSTRACT

A protein gel for encapsulation and controlled delivery of bioactive compounds is provided. These specific bioactive compounds may include but are not limited to vitamins, minerals, medications, probiotics and other bioactive supplements; and the amounts and/or dosages of the bioactive agents and ingredients may be adjusted to properly suit adults, children, or elderly dependent upon the end-use. This protein gel may act as both a pharmaceutical and nutraceutical, and may contain the active therapeutic compound, such as an antibiotic, chemotherapeutic, or pain medication in conjunction with specific nutrients that may act synergistically with the medication to enhance efficacy and overall health of the patient. The gel may be consumed alone or incorporated into food products such as cereal-type bars or mixed into biscuits, cookies, and the like.

### BACKGROUND

Delivering drags to patients in a safe, effective, and compliant manner is a major challenge for the treatment of many types of disease. The ability of drugs to reach target tissues from the point of oral administration can be limited by multiple barriers including enzymatic and acidic degradation in the stomach, absorption across the intestinal epithelium, hepatic clearance, and nonspecific uptake. Effective oral dosing to achieve high concentrations of drugs within specific tissues while minimizing systemic toxicity can present a significant challenge. Conventional polymeric drag delivery systems such as implants, injectable microspheres, and patches are used by tens of millions of people annually, yet often produce a sharp initial increase in concentration to a peak above the therapeutic range, followed by a fast decrease in concentration to a level below the therapeutic range.

Additionally, noncompliance with oral medication is a leading cause of hospitalizations. Oral medications such as liquids, powders, and capsules pose inherent risks and disadvantages. When administering liquid medications, one often uses a small dosage cup or syringe. The variation in accuracy of these dosage cups in combination with patient error can often result in improper dosage of the medication. Similar issues arise with the use of a powdered medication. The powdered medication must be reconstituted in a liquid before administration, resulting in the aforementioned dosing problems. Additionally, these liquids and reconstituted medications have a short shelf-life and expire quickly, resulting in the waste and disposal of vast quantities of medication.

Capsules provide an easier, more accurate dosing; however, capsules may become lodged in the throat and pose a choking hazard, particularly for children and the elderly. Additional risks occur when taking a controlled-release tablet or capsule. One must be careful not to chew or crush the tablet, as this results in immediate release of the entire dosage of medication rather than the slow, controlled release over time. Non-compliance in this area of drug administration often results in hospitalization due to drug overdosing.

Nutritional and dietary supplements such as multi-vitamins and minerals, botanicals and herbal extracts have grown in popularity, as evidenced by the tremendous growth in the industry involved in their manufacture, production and distribution. Such supplements can be consumed in a variety of ways, the most common being in powder or capsule form.

The consumption of powders suffers from problems such as low solubility or dispersability in water or juice and unpleasant mouthfeel and taste. A disadvantage of supplements in capsule form is the fact that in order to achieve the recommended daily amount, one must take multiple capsules once, twice, or even three times a day. Additionally, many supplements are poorly absorbed into the body, and studies have shown that the amount and quality of active ingredient varies widely from capsule to capsule, even if the capsules are derived from the same product lot.

Due to the problems and disadvantages associated with current conventional drug and supplement therapy, it would be advantageous to provide an alternative oral delivery system for drugs, dietary supplements, and the like. Providing a gel-based oral therapy overcomes the problems associated with current administration methods such as poor solubility and absorption, short-shelf life and stability, and safety and ease of use.

Gel-based oral administration has been experimented with and described in the following publications, all incorporated herein by reference. U.S. Pat. No. 7,531,192 discloses a method of preparing and using an ingestible matrix delivery system; U.S. Pat. No. 8,092,853 describes a method of making a gel-type livestock feed; U.S. Pat. No. 4,883,660 describes a gel-base for pharmaceutical compositions; U.S. Pat. No. 8,529,939 describes a mucoadhesive drug delivery device comprising one or more biocompatible purified proteins combined with one or more biocompatible solvents and one or more mucoadhesive agents; U.S. Patent Application Publication No. 2013/0280334 discloses a self-assembled gel composition to encapsulate one or more agents; and U.S. Patent Application Publication No. 2001/0006671 discloses a method for suspending water insoluble materials in edible oils,

### SUMMARY OF THE INVENTION

One aspect of the present invention is to provide a protein gel for encapsulation and controlled delivery of bioactive compounds. These specific bioactive compounds may include but are not limited to vitamins, minerals, medications, probiotics and other bioactive supplements.

Another aspect of the present invention is to provide a nutritional medication that can be administered in a safe, effective manner. This nutritional medication may act as both a pharmaceutical and nutraceutical, and may contain the active therapeutic compound, such as an antibiotic, chemotherapeutic, or pain medication in conjunction with specific nutrients that may act synergistically with the medication to enhance efficacy and overall health of the patient.

Yet another aspect of the present invention is to provide a protein gel that can be used as a base carrier for functional ingredients as desired by the manufacturer. The specific functional ingredients may be chosen according to the designated end use such meal supplement or meal replacement, athletic training, mood or energy enhancement, etc.

Yet another aspect of the present invention is to provide a gel that may be consumed alone or incorporated into food products, thus providing a meal replacement option. For example, the gel may be packaged in a stick pack whereby the consumer can simply squeeze the gel directly into his or her mouth. Alternatively, the consistency or viscosity of the gel may be such that the gel may be inserted into cereal-type bars or mixed into biscuits, cookies, and the like.

In a further aspect, the protein gel base is provided such that amounts and/or dosages of the bioactive agents and ingredients may be adjusted to properly suit adults, children, or elderly dependent upon the end-use.

### DETAILED DESCRIPTION OF THE INVENTION

As the invention allows for various changes and numerous embodiments, particular embodiments will be described in detail in the written description. However, this is not intended to limit the present invention to particular modes of practice, and it is to be appreciated that all changes, equivalents, and substitutes that do not depart from the spirit and technical scope of the present invention are encompassed in the present invention. In the description of the present invention, certain detailed explanations of related art are omitted when it is deemed that they may unnecessarily obscure the essence of the invention.

The terms used in the present application are merely used to describe particular embodiments, and are not intended to limit the present invention. An expression used in the singular encompasses the expression of the plural, unless it has a clearly different meaning in the context. In the present application, it is to be understood that the terms such as “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Unless otherwise defined, all terms used herein, including technical or scientific terms, have the same meanings as those generally understood by those with ordinary knowledge in the field of art to which the present invention belongs. Such terms as those defined in a generally used dictionary are to be interpreted to have the meanings equal to the contextual meanings in the relevant field of art, and are not to be interpreted to have ideal or excessively formal meanings unless clearly defined in the present application.

As used herein, the terms “formulation” and “composition” and “component” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutieaily active agent,” “pharmaceutical,” “active ingredient” or “functional agents” variations thereof may be used interchangeably to refer to an agent or substance that has measurable specified or selected physiologic activity when administered to a subject in a significant or effective amount. Non-limiting examples include drugs, botanical extracts, enzymes, hormones, proteins, polypeptides, antigens, nutritional supplements such as fatty acids, antioxidants, vitamins, minerals, as well as other pharmaceutically or therapeutically useful compounds. The functional ingredients may include ingredients having active effects in dental or medical hygiene, bone health, digestive aid, intestinal protection, general nutrition, stress relief, etc.

The term “drug” or “medication,” as used herein, refers to a pharmacologically active substance that exerts a localized or systemic effect or effects on an animal. It is to be understood that the term “drug” is expressly encompassed by the present definition as many drugs and prodrugs are known to have specific physiologic activities. These terms of art are well known in the pharmaceutical and medicinal arts.

The terms “botanical extract” and “herbal supplement,” as used interchangeably herein, refer to a substance derived from a plant source. Non-limiting examples may include echinacea, Siberian ginseng, ginkgo biloba, kola nut, goldenseal, gotu kola, schizandra, elderberry, St. Johns Wort, and valerian.

The term “nutritional supplement” as used herein refers to a substance that exerts a physiological effect on an animal. Typically, nutritional supplements fulfill a specific physiological function or promote the health and well-being of the consumer.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals. The term “animal” as used herein includes, but is not limited to, mammals including humans, birds and reptiles.

As used herein, “blood level” may be used interchangeably with terms such as blood plasma concentration, plasma level, plasma concentration, serum level, serum concentration, serum blood level and serum blood concentration. As used herein, “oral dosage form” and the like refers to a formulation that is ready for administration to a subject through the oral route of administration. Examples of known oral dosage forms, include without limitation, tablets, caplets, powders, pellets, granules, beads and mini tablets and combinations thereof etc. Such formulations also include multilayered tablets wherein a given layer may represent a different drug. In some aspects, granules, powders, pellets, minitablet, or nanoparticles may be coated with a suitable polymer/fats/waxes/emulsifiers/carbohydrates or a conventional coating material to achieve, for example, greater stability in the oral cavity, gastrointestinal tract, to achieve the desired rate of release, or to improve taste. Tablets and caplets may be scored to facilitate division of dosing. Alternatively, the dosage forms of the present invention may be unit dosage forms wherein the dosage form is intended to deliver one therapeutic dose per administration. Particular embodiments or groups of embodiments may be expressly limited to subsets of these dosage forms.

As used herein, “packet” or “stick pack” refers to a small, sealed packet containing a quantity of material, which is a single-use or unit dose quantity.

As used herein, an “effective amount” or a “therapeutically effective amount” of a drug or active ingredient refers to a sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors.

As used herein, “pharmaceutically acceptable earner” and “carrier” may be used interchangeably, and refer to any inert and pharmaceutically acceptable material that has substantially no biological activity, and makes up a substantial part of the formulation. The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would he the same as if it completely lacked particles. In other words, a composition that is “substantially free of” an ingredient or element may still actually contain such item as long as there is no measurable effect thereof. As used herein, the term “about” is used to provide flexibility to a numerical range endpoint by providing that a given value may be “a little above” or “a little below” the endpoint.

One aspect of the present invention is to provide a protein gel for encapsulation and controlled delivery of bioactive compounds. In a preferred embodiment, the main components of the protein gel base may include: water, preservatives, whey protein isolate, amino acids in peptide form, and a thickening agent. It is preferable to add flavoring as well to make the gel more palatable and enjoyable to consume.

The protein gel base described above may also include essential vitamins, minerals, and fatty acids thus forming a fully balanced nutrient gel to which additional bioactive compounds may be added as desired by the manufacturer. The preferable main components of the gel are described below.

### Whey Protein

In a preferred embodiment, the protein gel is formulated from whey protein. Whey is liquid by-product of the cheese making process which can be further processed into spray dried products, for instance, whey protein concentrates (WPC), whey protein isolates (WPI) or whey protein hydrolysates (WPH). As an important staple in the food industry, whey protein and its components have been subjected to in-depth characterization and study, though primarily as they relate to food science and engineering.

Characteristics of different types of whey protein are related to the chemical composition and processing technique. Whey proteins are widely used as important ingredients in different foods due to their unique nutritional and functional properties. Processing steps can alter the characteristics of whey protein products which can result in the modification of whey protein structure and functionality (i.e. foaming, emulsification, gelation). Whey protein is heat sensitive so thermal denaturing can he done at low temperatures, making thermal curing of protein solutions straight-forward. Added calcium ions participate in cross-linking, hydrogen bonding, and hydrophobic interactions on cooling, thus tightening the network and forming a strong matrix.

The protein gel described herein is preferably composed of a whey protein isolate. Numerous studies have determined the correlation between protein concentration and mechanical and rheological properties of whey protein isolate gels using a variety of conditions, fabrication methods, and gel compositions. The components of the whey protein isolate mixture are well characterized both in structure and in sequence, and its gelling properties have been extensively studied and are favorable for the current application. Information on whey protein solutions and gels at low concentrations is known primarily as it relates to food science. Although it is preferable to use whey protein isolate, it is understood that any suitable protein substrate may be used.

### Amino Acids

Another aspect of the present invention is using a whey protein isolate in conjunction with amino acids in a peptide form to create the protein gel base. It is preferable to use a collagen hydrolysate as the peptide source; although it is to be understood that any suitable source of peptides or free amino acids may be used. Collagen is one of the most abundant proteins in the body and is the main structural protein found in bone, skin, muscle, and other connective tissues. Upon hydrolysis, the collagen is broken down into peptides. These peptides are lower in molecular weight than the intact protein and are thought to be easily digested and have a better absorption and bioavailability than both the free amino acids and the whole protein.

In the present invention, it is preferable to have a ratio of the whey protein isolate to collagen hydrolysate wherein the units of whey protein isolate are greater than the units of collagen hydrolysate. Experimentation for the present invention has found that the preferred ratio range of whey protein isolate to collagen hydrolysate is 50:50 to 90:10, more preferably 60:40 to 80:20, and most preferably 70:30. It is found that this ratio range provides the best taste, consistency, texture, and bioavailability while still remaining feasible for manufacturing.

### Active Ingredients

The amount of active ingredient in the preparation is in the range of 0.1 mg to 10 g. Preferred prophylactic or therapeutic active ingredients contemplated for use in the present inventive subject matter are, without limitation, guaifenesin, mesalamine, diltiazem, metoprolol, balsalazide, aspirin, benzocaine, diphenhydramine, acetaminophen, ibuprophen and mixtures thereof. Preferred prophylactic or therapeutic active ingredients contemplated for use in the present inventive subject matter are antibiotics, which may be selected from the group consisting of amoxicillin and clavulanate potassium, ciprofloxacin HCl, azithromycin, clarithromycin, sterile ceftriaxone sodium, cefuroxime axetil, imipenem cilastatin, levofloxacin, ceftazidime, ampicillin sodium and sulbactum sodium, cefaclor, amoxicillin, cefdinir, roxithromycin, sterile cefotaxime sodium, vancomycin, piperacillin sodium and tazobactum sodium, momiflumate, flomoxef sodium, cefotiam dihydrochloride, ofloxacin, mupirocin calcium, vancomycin HCl, teicoplanin, cefadroxil monohydrate, sulbactam cefoperazone, meropenem, ofloxacin, cephalexin, cefepime HCl, cefuroxime sodium, minocycline HCl, cefaclor, cefazolin, trimethoprim and sulfamethoxazole, norfloxacin, trovafloxacin, cefpodoxime proxetil, cefdinir, cellxime, panipenem, ceftibuten, levofloxacin, cefoxopran HCl, amikacin sulfate, aztreonam, minocycline HCl, ticarcillin disodium or mixtures thereof.

### Inactive Ingredients

Many of the active ingredients listed above have unpalatable tastes. Taste-masking of compositions with those unpalatable active materials is well-known in the art. The active ingredient may coated with a suitable polymer/fats/waxes/emulsifiers/carbohydrates. The use of flavors and sweeteners to mask the unpalatability of the active materials is also well-known. Thus, other materials which can be incorporated into composition include flavors, colors and sweeteners. Importantly, it is possible to incorporate high levels of flavors, sweeteners and other taste-masking agents, making the compositions more palatable when undesirable tastes accompany the active materials.

Taste masking may be chosen from natural and synthetic flavor liquids. Flavors useful include, without limitation, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Taste masking of the active ingredients can be done using the well-known processes in the art such as fiuidization, spray drying, spray congealing, complex co-acervation, resin complexation, matrix granulation using carbohydrates, resins, polymers, waxes & fats.

Taste enhancers may be chosen from natural and synthetic flavor liquids. Flavors useful include, without limitation, volatile oils, synthetic flavor oils, flavoring aromatics, oils, liquids,

### Process

The preferred process for making the protein gel is described herein; while specific amounts and specific ingredients are outlined below in the Examples section. The first step includes adding water to a mixing apparatus such as Silverson Flash Mix. The precise volume of water added may be determined by the volume of the additional ingredients that are to be dissolved into solution, as well as the desired density, viscosity, and consistency of the final product. Enough water should be added to dissolve all the ingredients into a solution; any additional water may be added to achieve the desired thickness of the gel. For a thicker, more viscous gel, less water may be used. For a thinner, more fluent gel, more water may be used. Preservatives may be added next. Any food preservative may be suitable; some examples include potassium sorbate and sodium benzoate. The whey protein isolate may be added in combination with amino acids, whereby the units of whey protein isolate preferably are greater than the units of collagen hydrolysate. One example of whey protein isolate is BevWise A-100W, manufactured by Glanbia Nutritionals. It is preferable to add these protein components early in the mixing process to allow for complete and adequate dispersion of these compounds in the solution. Also, it is best to add a foam suppressing agent such as Antifoam during this step, as the whey protein has a tendency to foam in solution. As discussed previously, it is preferable to add the amino acids in the form of peptides such as a collagen hydrolysate, one example being Solugel 5000, manufactured by PB gelatins. The next step in the mixing process is to add a thickening agent such as food starch. Similar to the amount of water added, the amount of thickening agent added may be determined by the desired thickness of the gel. The thickening agent, along with the whey protein isolate and collagen hydrolysate, will solidify during the heating process (occurring later) thus forming the gel base. At this step in the process, optional sweeteners, flavorings, and/or colorings may be added. Sweeteners may include sugar (sucrose), syrups, artificial sweeteners such as stevia, and the like. Flavorings may be any suitable flavor as desired such as orange, banana, peach, vanilla, bubble gum, etc. Typical food-based colorants may be added to make the gel more visually appealing.

After adding and mixing the aforementioned components that formulate the gel base, the desired bioactive compounds, medications, herbal supplements, and/or functional ingredients may be added. The type and amount of these compounds is dependent upon end use. For example, if the gel is being designed as a pain reliever, it will contain the appropriate pain medication, such as acetaminophen. The chosen dose will be dependent upon the consumer for which the gel is designed. For example, an acetaminophen dose will be smaller (for example, 10-15 mg/kg/dose) if the gel is designed for children; alternatively, the dose will be larger (for example 325-1000 mg) if the gel is appropriate for adults. Another example may include the gel being designed as a prenatal supplement. A prenatal gel may contain vitamins and minerals along with higher amounts of folic acid (at least 400 mcg is recommended) and DHA (at least 300 mg) appropriate for pregnant or nursing women in conjunction with other supplements such as ginger which is thought to help alleviate morning sickness. The percent daily values or dosage amounts for these bioactive compounds, drugs, etc. are determined and recommended by physicians and experts, and one skilled in the art would know how to calculate the appropriate amounts of these compounds to add to the mixture based on these recommended values or dosages.

Many of these types of compounds are light sensitive and some may be oxygen sensitive, for this reason, it is best to add these components last in the mixing process, thus minimizing the exposure to air and light. Additional steps may be taken to reduce exposure to light and air, such as maintaining an enclosed mixing apparatus and nitrogen flushing the gel to facilitate the removal of any oxygen that might be present.

It is preferable to perform the mixing process at room temperature, although lower temperatures may be used as well, provided the components remain flowable. The use of relatively low temperatures in the preparation of the gel, when compared to typical manufacturing procedures using much higher temperatures, ensures that the functional ingredients are not degraded by excessive heat. Once all the necessary and desired compounds have been added, the temperature may be raised to a set point necessary to initiate solidification of the gel. Most protein gels are prepared by heating, and the preferred temperature set point is at or around 185 degrees Fahrenheit. Thermal gelation of whey proteins is a two-step mechanism. The first step involves an initial denaturation and unfolding of whey protein molecules, followed by rearrangements and aggregation of functional groups which become available for intermolecular interactions under appropriate conditions, resulting in a three-dimensional gel network. This phenomenon is important in food industry due to strong effect on rheological and textural properties of food.

The delivery systems of the present invention can be formulated such that the gel has a final pH in the range of about 2.5 to about 8.5. However, it should be noted that the pH may vary based on specific functional ingredients and bioactive compounds. In one preferred embodiment, the gel has a final pH of between about 3.0 and 3.4. Acidic pH is known in the art to promote degradation of certain functional ingredients. For delivery systems formulated to deliver functional ingredients which are sensitive to, or reactive at, acidic pH, the final pH of the gel is neutral to mildly basic. By neutral to mildly basic pH it is meant that the final pH is between about 6.0 and about 8.5.

For those functional ingredients that are more stable in acidic form, such as trimethylglycine, or functional ingredients which may react with other components at neutral pH such as glucosamine hydrochloride, the pH of the gel delivery system may have a final pH below neutral. By below neutral, it is meant that the final pH is between about 2.5 and about 6.0. In another embodiment of the present invention, therefore, the delivery systems are formulated such that the gel has a final pH between about 3.0 and about 6.0 and thus are suitable for delivery of functional ingredients that are stable at acidic pH and/or interact with other components at neutral pH.

Due to the substantially uniform and complete dispersion of the functional ingredients within the gel, the current delivery system is suitable for preparation in large batches and then divided into sub-units. Such division would not be possible with other delivery systems in which the functional ingredients are not evenly dispersed.

Providing an oral therapy in the proposed gel form allows for a much more stable product with increased shelf-life, easier portability and storage, and safety and ease of administration. The final gel may packaged and consumed alone or may be incorporated with other food products. For example, the gel may be packaged in single serving or single dose gel packs or stick pack whereby one may squeeze the gel directly into his or her mouth. Or, the gel may be manufactured to be inserted into a cereal bar, spread on bread, or mixed into a cookie, biscuit, or other similar and appropriate food items. By manufacturing the gel to include nutritional supplements such as vitamins and minerals and to be consumed with a cereal bar or whole grain biscuit, the final product provides a meal replacement or meal substitute.

Furthermore, the viscosity of the gel base may be adjusted as desired by the addition or subtraction of both water and thickening agent. The final gel may be a thinner consistency similar to maple syrup, a thicker consistency similar to a jelly, or any consistency in-between. It yet another embodiment, it is contemplated that the gel may be poured into a mold, squeezed onto a surface, or spread out in a desired shape. By removing a pre-determined amount of the water content, the resulting gel resembles more of a gummy texture that may be chewed.

The delivery systems according to the present invention are suitable for administration to both human and non-human animals. One skilled in the art will appreciate that each delivery system can be formulated differently according to the type of animal to which it is to be administered. For example, for administration to an animal such as a cat or a dog, meat or fish-based flavors may be added. For administration to a human, the delivery system may be formulated, for example, as a confectionery using fruit-based or other flavors. The delivery systems are especially suited for oral administration due to their palatability. Additionally, due to the highly portable format, the delivery systems are simple and convenient to administer and to consume for both humans and other animals.

The delivery systems of the present invention can be tailored for specific purposes, thus the delivery systems can be formulated with specific combinations of functional ingredients in order to produce specific physiological effects. For example, a drug delivery system can be formulated to contain certain combinations of drugs or diagnostic agents. Other delivery systems can be formulated with combinations of functional ingredients for example to promote endurance, promote cardiovascular health, control fat and/or cholesterol, promote healthy joints, maintain or improve bone density, enhance cellular antioxidant capacity, control appetite, athletic enhancement, or any healthful effect as desired. Combinations of ephedrine and caffeine are known in the art to produce a thermogenic effect and can be included in a thermogenic delivery system.

Similarly combinations of Ginkgo biloba and gotu kola are used for memory enhancement and can be included in a memory enhancement delivery system. Other non-limiting examples include, delivery systems formulated with combinations of functional ingredients to promote energy, increase endurance, promote weight loss, promote muscle enhancement, improve digestion, or help prevent colds or fight infection.

The compositions are “storage stable”, meaning that the compositions are stable in the absence of special handling procedures. The inventive compositions are stable both prior to packaging and after packaging. Importantly, the inventive compositions maintain their stability and integrity without refrigeration and without humidity controls being implemented during handling, packaging and storing of the products. Additionally, since the compositions exhibit increased integrity and stability, the compositions can be used in most of the current economical packages suitable for a global environment. Further, high temperatures are not needed when processing the packaging and sealing.

The formulas for an exemplary list of products are shown below. Without further description, it is believed that one of ordinary skill in the art can, using the preceding description and the following illustrative examples, make and use the present invention. The following examples are given to illustrate the present invention. It should be understood that the invention is not to be limited to the specific conditions or details described in the examples.

### EXAMPLE 1

### Sugar-Free Nutrient Gel

1. Add the contents of Group A into the tank of a Silverson Flash Mix, and turn mixer on.

### Group A

| | | | Units of |
|---|---|---|---|
| Ingredient | % w/w | Quantity | Measurement |
| Purified Water | 82.5238 | 816.241 | gal |
| SUBTOTAL | 82.5238 | 816.241 | gal |

2. Add the contents of Group B (preservatives) from the top of the tank.

### Group B

| | | | Units of |
|---|---|---|---|
| Ingredient | % w/w | Quantity | Measurement |
| Potassium Sorbate | 0.0796 | 2.879 | kg |
| Sodium Benzoate | 0.0769 | 2.879 | kg |
| SUBTOTAL | 0.1538 | 5.758 | kg |

3. Add whey protein isolate (BevWise A-100W) slowly into the Silverson Flash Mix. After 1-2 bags of the BevWise A-100W have been added, add Supressor 3569 (Antifoam) to control foam build-up in the product. Stop the mixer and turn on the impeller of the tank for approximately 3-7 minutes. Turn off the impeller and turn the mixer back on. Continue adding BevWise A-100W followed by collagen hydrolysate (Solugel 5000).

### Group C

| | | | Units of |
|---|---|---|---|
| Ingredient | % w/w | Quantity | Measurement |
| BevWise A-100 W | 5.7231 | 214.273 | kg |
| Solugel 5000 | 2.1846 | 81.791 | kg |
| Suppressor 3569-Antifoam | 0.0769 | 2.879 | kg |
| SUBTOTAL | 7.9846 | 298.943 | kg |

4. Slowly add the contents of Group D (thickening agent, flavor) into the Silverson Flash Mix.

### Group D

| | | | Units of |
|---|---|---|---|
| Ingredient | % w/w | Quantity | Measurement |
| Pure Gel B994 | 3.2308 | 120.961 | kg |
| Cream Flavoring | 0.4615 | 17.279 | kg |
| SUBTOTAL | 3.6923 | 138.240 | kg |

5. Slowly add the contents of Group E (vitamins, minerals, artificial sweeteners) into the Silverson Flash Mix.

### Group E

| | | | Units of |
|---|---|---|---|
| Ingredient | % w/w | Quantity | Measurement |
| Calcium Lactate-Gluconate | 0.6923 | 25.920 | kg |
| Magnesium Citrate Malate | 0.4615 | 17.279 | kg |
| Truvia Stevia RA80 | 0.0462 | 1.730 | kg |
| Sucralose | 0.0769 | 2.879 | kg |
| Selenium 1% | 0.0031 | 0.116 | kg |
| Zinc 15% | 0.0277 | 1.037 | kg |
| Iron 15% | 0.0311 | 1.239 | kg |
| Potassium Iodide 3% | 0.0038 | 0.124 | kg |
| Beta Carotene | 0.1231 | 4.609 | kg |
| SUBTOTAL | 1.4677 | 54.951 | kg |

6. Add the contents of Group F (vitamins, minerals, flavoring) into the Silverson Flash Mix,

### Group F

| | | | Units of |
|---|---|---|---|
| Ingredient | % w/w | Quantity | Measurement |
| Taste Modifier | 0.3077 | 11.520 | kg |
| Niacin USP | 0.0217 | 0.812 | kg |
| Pyridoxine | 0.0022 | 0.0824 | kg |
| HCl USP (Vit B6) | | | |
| Thiamine | 0.0025 | 0.0936 | kg |
| Hydrochloride USP | | | |
| Riboflavin USP | 0.0017 | 0.0636 | kg |
| Vitamin B12 UST | 0.000005 | 0.0001728 | kg |
| Cyanocobalamin | | | |
| Calcium D | 0.0108 | 0.4044 | kg |
| Panthothenate-UST | | | |
| Folic Acid USP | 0.0005 | 0.0187 | kg |
| Vitamin K1 | 0.0011 | 0.0412 | kg |
| (5% Phytonadione) | | | |
| Vitamin A | 0.0169 | 0.6327 | kg |
| Palmitate Type 250 | | | |
| Vitamin D3 Type 100 | 0.0052 | 0.1947 | kg |
| Vitamin E 700 | 0.0308 | 1.153 | kg |
| DATocopheryl | | | |
| Acetate | | | |
| Biotin 1% UST | 0.0037 | 0.139 | kg |
| SUBTOTAL | 0.4048 | 15.156 | |

7. Add the contents of Group G from the top of the tank.

### Group G

| | | | Units of |
|---|---|---|---|
| Ingredient | % w/w | Quantity | Measurement |
| Potassium Phosphate Monobasic | 0.4000 | 14.976 | kg |
| Malic Acid | 1.2308 | 46.081 | kg |
| SUBTOTAL | 1.6308 | 61.057 | kg |

8. Add the contents of Group H (flavorings and fatty acids) into the Silverson Flash Mix.

### Group H

| | | | Units of |
|---|---|---|---|
| | | | Measure- |
| Ingredient | % w/w | Quantity | ment |
| Flavor “A” | 0.6154 | 23.041 | kg |
| Flavor “B” | 0.2308 | 8.641 | kg |
| Phosphoric Acid | 0.6923 | 25.920 | kg |
| Omega Emulsion (5%) | 0.5385 | 20.161 | kg |
| OTEC 250 CL-K | | | |
| Ascorbyl Palmitate | 0.0615 | 2.303 | kg |
| SUBTOTAL | 2.1385 | 80.065 | kg |

9. After all ingredients from Group H have been added, turn the mixer off, and turn the impeller on. Leave the impeller running for at least 30 minutes to ensure proper blending.
10. Once the formulation appears to be well-mixed, turn the impeller off. Add Group I and turn the impeller on and let run for 5 minutes.

### Group I

| | | | Units of |
|---|---|---|---|
| Ingredient | % w/w | Quantity | Measurement |
| Ascorbic Acid | 0.0038 | 0.142 | kg |
| SUBTOTAL | 0.0038 | 0.142 | kg |
| TOTAL | 100.0001 | | |

11. Leave the impeller of the blending tank running and check the pH of the product. If the pH is less than or equal to 3.34, proceed with high temperature, short time (HTST) flash pasteurization with a set point temperature of 185 degrees F and a flow rate of 8 gallons per minute.
12. Once 185 degrees F is reached, hold the product for solidification for approximately 45 seconds, then collect in clean drums lined with two plastic bags. The gel discharge temperature is approximately 110-120 degrees F.
13. Nitrogen flush the drums before and after filling with gel to minimize the oxidation of the ascorbic acid. Be sure to place lids on the drums immediately after filling to minimize exposure to light.
14. Record the temperature of the gel prior to sub-dividing into packaging.
15. Within 24 hours of filling the drums, check the pH of the gel to ensure a pH of less than or equal to 3.34 prior to distribution and/or consumption.

While the spirit of the invention has been described in detail with reference to particular embodiments, the embodiments are for illustrative purposes only and do not limit the invention. It is to be appreciated that those skilled in the art can change or modify the embodiments without departing from the scope and spirit of the invention.

We claim:

1. A gel-based oral delivery composition comprising whey protein isolate, collagen hydrolysate, and a thickening agent, whereby the ratio of whey protein isolate to collagen hydrolysate ranges from 50:50 to 90:10.

2. The gel-based oral delivery composition of claim 1, whereby the ratio of whey protein isolate to collagen hydrolysate ranges from 60:40 to 80:20.

3. The gel-based oral delivery composition of claim 1, whereby the ratio of whey protein isolate to collagen hydrolysate is 70:30.

4. The gel-based oral delivery composition of claim 1, further including a flavoring agent, a sweetener, and a food preservative.

5. The gel-based oral delivery composition of claim 1, further including at least one active ingredient in an amount ranging from 0.1 milligrams to 10 grams.

6. The gel-based oral delivery composition of claim 5, whereby said active ingredient is selected from the group consisting of guaifenesin, mesalamine, diltiazem, metoprolol, balsalazide, aspirin, benzocaine, diphenhydramine, acetaminophen, ibuprophen and mixtures thereof.

7. The gel-based oral delivery composition of claim 5, whereby said active ingredient is an antibiotic.

8. The gel-based oral delivery composition of claim 7, whereby said antibiotic selected from the group consisting of amoxicillin-potassium clavulanate, ciprofloxacin HCl, azithromycin, clarithromycin, sterile ceftriaxone sodium, cefuroxime axetil, imipenem cilastatin, levofloxacin, ceftazidime, ampicillin sodium and sulbactum sodium, cefaclor, amoxicillin, cefdinir, roxithromycin, sterile cefotaxime sodium, vancomycin, piperacillin sodium and tazobactum sodium, morniflumate, flomoxef sodium, cefotiam dihydrochloride, ofloxacin, mupirocin calcium, vancomycin HCl, teicoplanin, cefadroxil monohydrate, sulbactum cefoperazone, meropenem, ofloxacin, cephalexin, cefepime HCl, cefuroxime sodium, minocycline HCl, cefaclor, cefazolin, trimethoprim and sulfamethoxazole, norfloxacin, trovafloxacin, cefpodoxime proxetil, cefdinir, cefixime, panipenem, ceftibuten, levofloxacin, cefoxopran HCl, amikacin sulfate, aztreonam, minocycline HCl, ticarcillin disodium, and mixtures thereof.

9. The gel-based oral delivery composition of claim 1, further including at least one nutritional supplement.

10. The gel-based oral delivery composition of claim 9, wherein said nutritional supplement is provided in such an amount that the percent daily value of the supplement in a single serving of the gel-based oral delivery system is at least 0.5%.

11. The gel-based oral delivery composition of claim 9, whereby said nutritional supplement is selected from the group consisting of vitamin A, vitamin C, vitamin D, vitamin E, vitamin K, thiamine, riboflavin, niacin, pantothenic acid, vitamin B6, biotin, folic acid, vitamin B12, calcium, iodine, zinc, copper, iron, phosphorus, magnesium, selenium, manganese, omega-3 fatty acid, omega-6 fatty acid, and mixtures thereof.

12. The gel-based oral delivery composition of claim 1, whereby said composition is packaged in a single serving pouch, such that said composition may be consumed directly from said pouch.

13. A nutritional meal replacement comprising:
a gel base comprising whey protein isolate, collagen hydrolysate, and a thickening agent, whereby the ratio of whey protein isolate to collagen hydrolysate ranges from 50:50 to 90:10;
a whole grain cereal bar; and
said gel base being packaged in a single serving stick pack;
said single serving stick pack and said whole grain cereal bar packaged together in a single package such that said gel base and said cereal bar may be consumed simultaneously.

14. The nutritional meal replacement of claim 13, whereby said gel base further includes at least one nutritional supplement selected from the group consisting of vitamin A, vitamin C, vitamin D, vitamin E, vitamin K, thiamine, riboflavin, niacin, pantothenic acid, vitamin B6, biotin, folic acid, vitamin B12, calcium, iodine, zinc, copper, iron, phosphorus, magnesium, selenium, manganese, omega-3 fatty acid, omega-6 fatty acid, and mixtures thereof.

15. A process of making a single serving gel-based oral delivery composition, comprising the steps of:
mixing a solution of whey protein isolate and purified water;
adding collagen hydrolysate to the solution in an amount such that the whey protein isolate and collagen hydrolysate are in a ratio range of 50:50 to 90:10;
adding a thickening agent to the solution of water, whey protein isolate, and collagen hydrolysate;
blending the solution for at least 30 minutes;
ensuring that the pH of the solution is in the range of 2.5 to 8.5;
performing high temperature, short time flash pasteurization with a set point temperature of at least 185 degrees F and a flow rate of 8 gallons per minute;
holding the solution until solidification of the solution into a gel occurs; and
collecting the gel in receptacles for distribution.

16. The process of claim 15, further including the step of adding at least one nutritional supplement selected from the group consisting of vitamin A, vitamin C, vitamin D, vitamin E, vitamin K, thiamine, riboflavin, niacin, pantothenic acid, vitamin B6, biotin, folic acid, vitamin BI2, calcium, iodine, zinc, copper, iron, phosphorus, magnesium, selenium, manganese, omega-3 fatty acid, omega-6 fatty acid, and mixtures thereof.

17. The process of claim 15, further including the step of adding at least one active ingredient selected from the group consisting of guaifenesin, mesalamine, diltiazem, metoprolol, balsalazide, aspirin, benzocaine, diphenhydramine, acetaminophen, ibuprophen, amoxicillin-potassium clavulanate, ciprofloxacin HCl, azithromycin, clarithromycin, sterile ceftriaxone sodium, cefuroxime axetil, imipenem cilastatin, levofloxacin, ceftazidime, ampicillin sodium and sulbactam sodium, cefaclor, amoxicillin, cefdinir, roxithromycin, sterile cefotaxime sodium, vancomycin, piperacillin sodium and tazobactum sodium, morniflumate, flomoxef sodium, cefotiam dihydrochloride, ofloxacin, mupirocin calcium, vancomycin HCl, teicoplanin, cefadroxil monohydrate, sulbactum cefoperazone, meropenem, ofloxacin, cephalexin, cefepime HCl, cefuroxime sodium, minocycline HCl, cefaclor, cefazolin, trimethoprim and sulfamethoxazole, norfloxacin, trovafloxacin, cefpodoxime proxetil, cefdinir, cefixime, panipenem, ceftibuten, levofloxacin, cefoxopran HCl, amikacin sulfate, aztreonam, minocycline HCl, ticarcillin disodium, and mixtures thereof.

18. The process of claim 15, further including the step of adding sweeteners and flavorings to the solution after adding the thickening agent.

19. The process of claim 15, further including the step of distributing the gel in single serving stick packs.
